(19) United States
(12) Patent Application Publication
(10) Pub. No.: US 2013/0108584 A1
(43) Pub. Date: May 2, 2013

(54) RECOMBINANT VECTOR FOR PRODUCING AND SECRETING AMINO ACID SEQUENCES OF INTEREST BY PROPIONIBACTERIA AND APPLICATIONS THEREOF

(71) Applicants: Gwenael Jan, Rennes (FR); Marie-Thérèse Dimanche-Boitrel, Melesse (FR); Hélène Falentin, Mordelles (FR)

(72) Inventors: Gwenael Jan, Rennes (FR); Marie-Thérèse Dimanche-Boitrel, Melesse (FR); Hélène Falentin, Mordelles (FR)

(73) Assignees: UNIVERSITE DE RENNES 1, Rennes (FR); INSTITUT NATIONAL DE LA RECHERCHE AGRONOMIQUE, Paris (FR); INSTITUT NATIONAL DE LA SANTE ET DE LA RECHERCHE MEDICALE (INSERM), Paris (FR)

(21) Appl. No.: 13/698,213

(22) PCT Filed: May 12, 2011
(86) PCT No.: PCT/EP11/57718

§ 371 Date: Nov. 15, 2012

(30) Foreign Application Priority Data
May 12, 2010 (FR) 1053750

(51) Int. Cl. C12N 15/74

(52) U.S. Cl. CPC C12N 15/746
USPC 424/93.2; 435/320.1; 435/252.3; 435/69.1; 514/44 R

(57) ABSTRACT

The present invention relates to a recombinant vector for expressing and secreting, by a propionibacterium, one or more amino acid sequences of interest, wherein said vector comprises at least: under the control of at least one suitable promoter, at least one nucleotide sequence coding for a propionibacteria signal peptide and, in translational fusion with said nucleotide sequence, one or more nucleotide sequences coding for said amino acid sequence or sequences of interest. The invention further relates to the uses of such a vector in the pharmaceutical field or for the large-scale production of peptides or proteins of interest.

The present invention relates to the field of genetic engineering applicable in particular in the pharmaceuticals, chemicals, agri-foods and cosmetics industries, etc.

More precisely, the present invention relates to a recombinant vector for expressing and secreting, by a propionibacterium, one or more amino acid sequences of interest, wherein said vector comprises at least:
under the control of at least one suitable promoter,
at least one nucleotide sequence coding for a propionibacteria signal peptide and, in translational fusion with said nucleotide sequence,
one or more nucleotide sequences coding for said amino acid sequence or sequences of interest.

The invention also relates to the uses of such a vector in the pharmaceutical field or for the large-scale production of peptides or proteins whose activity is of interest in industries as diverse as pharmaceuticals, chemicals, agri-foods, cosmetics, etc.

There are today a large number of methods and means for the large-scale production of peptides and/or proteins. With traditional chemical synthesis, which is hardly suitable for large-scale production of proteins comprising several dozen amino acids, the generally preferred method is synthesis in vivo, that is, in living biological systems. Many commercial enterprises in France and elsewhere have made a principal business of said production, and thus propose technologies based on genetic engineering and biotechnology to provide industrial quantities of peptides and/or proteins, preferably active and purified. Depending on needs, it is now possible to have recourse to various living systems such as bacteria (e.g., Escherichia coli), yeasts (e.g., Saccharomyces cerevisiae, Pichia pastoris), insect cells (baculovirus, Sf9, Sf21, etc.), plant cells and mammalian cells (e.g., CHO, HEK, COS, etc.).

Nevertheless, there still does not exist, at present, a “perfect” living system, that is to say, a system that has universal application (in particular to express any protein and to produce any quantity) and is at the same time simple to make use of, powerful, reliable and affordable.

The present invention aims precisely at mitigating these shortcomings while proposing for the first time the use of propionibacteria as living systems for producing and secreting recombinant peptides and proteins, especially from eukaryotic origin.

Propionibacteria (PB) and, more particularly, dairy propionibacteria (DPB) (notably Propionibacterium freudenreichii) have a particular metabolism which rests on the anaerobic conversion of sugars or lactic acid into short-chain fatty acids (SCFA), such as propionate, acetate and butyrate SCFA. These bacteria are used principally as ripening starter for cooked, pressed cheeses. Until now, few scientific teams or research and development laboratories have been interested in these bacteria. Several probiotic applications are known for said bacteria (for example, the Propiofidus® formulation marketed by Laboratoires Standa, France). They are indeed able to modulate the complex ecosystem of the colon in terms of microbial flora (Bougle et al., 1999) and enzymatic activities (Zarate et al., 2000).

The Inventors have recently cloned and sequenced the genome of the probiotic anaerobic firmicute bacterium Propionibacterium freudenreichii (Falentin et al., 2010, Plos One; Genbank accession No. FN806773). This bacterium has in particular cytotoxic properties with respect to colon cancer cells (Jan et al., 2002; Lan et al., 2007). Among other notable properties, it adheres to colonic epithelial cells and has no toxic effect on healthy cells (Lan et al., 2008).

Although they grow rather slowly, PB have the considerable advantage of being naturally able to secrete peptides and proteins in the extracellular medium, thus facilitating the recovery of said peptides and proteins without denaturation and without deterioration of the producing cells which can thus be advantageously recycled. These bacteria are very robust and adapt to particular media (such as media containing milk or milk derivatives, e.g., whey, and media containing molasses) that are possibly hostile to the growth and development of other living systems (presence in the medium of lactic acid, salt, etc.), and have good tolerance with respect to variations, changes or disturbances of the environmental conditions likely to occur during large-scale culture operations. Moreover, they can be described as “natural antifungals” because they naturally produce metabolites (for example, propionate) that inhibit the development of contaminating fungi. Furthermore, they are able to produce recombinant proteins of significant size (for example, proteins of more than 500 amino acids) and can even produce several different proteins simultaneously (for example, more than a dozen different proteins).

PB are thus completely suitable to serve as living “factories” for the large-scale production of recombinant peptides and proteins of interest, in particular in the context of in vitro or ex vivo processes or applications.

But their utility does not stop there. Indeed, in mammals, including humans, these bacteria are naturally able to target the intestine where their survival time can reach roughly two weeks, compared to that of lactic bacteria in particular which, although they are natural hosts of this ecosystem, is only two or three days on average. Thus, PB can also be used as tool for specific addressing or targeting in vivo for colon delivery of peptides and/or proteins of interest, in particular of therapeutic interest.

Some major advantages of PB over various other bacteria proposed so far for use in anti-tumoral therapy (e.g., in WO 01/25397 in the name of Vion Pharmaceuticals, Inc. and in WO 2009/111177 in the name of Mount Sinai School of Medicine of New York University) are that BP per se are an efficient and specific anti-tumoral agent that can safely be used in mammals, in particular in humans. This inherent property of BP can thus be further enhanced upon using BP to deliver therapeutic agents, such as drugs, to eradicate tumor cells while at the same time preventing damage to normal cells. To do so, BP do not need to be genetically attenuated or enhanced by genomic mutations as it is the case for other bacteria such as Clostridium, Salmonella, Listeria, and the like. BP eventually are a “super” anti-tumoral agent thanks to both their intrinsic anti-tumoral properties and their ability to efficiently, specifically and safely deliver other anti-tumoral drugs in order to kill cancer cells.

Thus, the present invention relates to a recombinant vector for expressing and secreting, by propionibacteria, one or more eukaryotic amino acid sequences of interest, comprising at least:
under the control of at least one suitable promoter,
at least one nucleotide sequence coding for a propionibacteria signal peptide and, in translational fusion with said nucleotide sequence,
one or more nucleotide sequences coding for said eukaryotic amino acid sequence or sequences of interest.

In the context of the invention, the terms “vector,” “plasmid vector” and “plasmid” are equivalent and are used in accordance with the usual meaning in the fields of molecular biology, genetic engineering and microbiology. Very briefly, a plasmid vector is a non-viral DNA molecule hosted by a cell, distinct from the natural chromosomal DNA of said host cell and capable of autonomous replication. The choice of vector and, more particularly, the origin of replication it carries thus depend on the host cell. According to the type of host cell, several copies of a vector and/or several different vectors can be hosted simultaneously. A vector according to the invention can possibly be carried by (or “integrated in” or “inserted in”) the chromosome of the host cell.

A “recombinant vector” is a plasmid obtained by traditional molecular biology and genetic engineering techniques, in which one or more exogenous nucleotide sequences have been inserted (or cloned). To simplify matters, the term “vector” as used herein is understood to relate to a recombinant vector.

Herein, a “host cell” is a PB, preferably a DPB, more preferably a DPB selected from the species Propionibacterium freudenreichii, P. jensenii, P. thoenii and P. acidipropionici. Also preferably, a host cell in accordance with the invention is P. freudenreichii, more particularly P. freudenreichii subsp. freudenreichii or P. freudenreichii subsp. shermanii. The most preferred host cell is P. freudenreichii subsp. shermanii.

In the context of the present invention, an “amino acid sequence” is selected from peptides and proteins, as well as fragments, analogs, derivatives and combinations of peptides and proteins. An “amino acid sequence” can thus be, according to its size, a peptide or a protein. Roughly speaking, “peptide” typically refers to a sequence of up to 50 amino acids, and “protein” or “polypeptide” refers to a sequence of more than 50 amino acids.

A protein or peptide “fragment” is a smaller peptide or protein whose amino acid sequence is included in that of the initial peptide or protein. A protein “fragment” could be a peptide, for example.

“Analog” refers to any modified version of an initial compound, in this case a protein or a peptide, wherein said modified version is natural or synthetic, and wherein one or more atoms, such as carbon, hydrogen or oxygen atoms, or heteroatoms such as nitrogen, sulfur or halogen, have been added or removed from the structure of the initial compound, so as to obtain a new molecular compound.

A “derivative” in the context of the invention is any compound that has a resemblance or a structural motif in common with a reference compound (in this case a protein or a peptide). This definition further includes, on the one hand, compounds that, alone or with other compounds, can be precursors or intermediate products in the synthesis of a reference compound, via one or more chemical reactions, and, on the other hand, compounds that can be formed from said reference compound, alone or with other compounds, via one or more chemical reactions. Thus, the term “derivative” covers at least protein and/or peptide hydrolysates, in particular tryptic hydrolysates, hydrolysate fractions and mixtures of hydrolysates and/or hydrolysate fractions. This definition also covers peptidomimetics or pseudopeptides, which are small molecules that mimic the bioactive properties of a reference peptide (Patch et al., 2002).

Moreover, the terms “analog” and “derivative” of a peptide or protein cover, for example, a peptide or a protein that is glycosylated or phosphorylated or has undergone any grafting of a chemical group.

To simplify matters, the term “protein” is used herein in the broad sense, wherein this generic term covers peptides and proteins as well as derivatives, fragments, analogs and combinations of peptides and proteins.

A “nucleotide sequence” or a “nucleic acid” according to the invention is in accordance with the usual meaning in the field of biology. These two expressions equally cover DNA and RNA, wherein the former can be genomic DNA, plasmid DNA, recombinant DNA or complementary DNA (cDNA), for example, and the latter can be messenger RNA (mRNA), ribosomal RNA (rRNA) or transfer RNA (tRNA). Preferably, the nucleotide and nucleic acid sequences of the invention are DNA.

For the sake of convenience, when the invention refers to “a” vector, “a” protein or “a” nucleotide or amino acid sequence, etc., it is understood that “a” or “the” also covers the use of several vectors, proteins, sequences, etc.

The recombinant vector of the present invention can “express” a protein. Indeed, it carries a nucleotide sequence which is transcribed and then translated in the host cell, to produce the protein in question. It is thus an “expression” or “production” or “synthesis” vector for said protein.

Moreover, the recombinant vector of the present invention can “secrete” a protein. Thus, the protein expressed from said vector is transported toward the outside of the host cell. The terms “secretion,” “transport toward the outside of the host cell,” “export” and “externalization” are equivalent herein and mean that the protein is expressed and then is exposed to the extracellular medium. Subject to this exposure, it can possibly remain anchored to the membrane of the host cell. However, preferably, the protein expressed by the host cell is “released” (or “delivered” or “salted out”) in the extracellular medium.

The “extracellular medium” is, as its name indicates, the medium surrounding the PB. It is understood herein that the expressions “extracellular medium,” “exterior medium,” “external medium,” “surrounding medium” and “environment” are synonymous. To simplify matters, the term “medium” will be used below. In embodiments of the present invention, the PB is grown in vitro or ex vivo (use of the PB as a “factory” to produce the proteins of interest). In this case, the extracellular medium is the culture medium. It can also be the supernatant of the culture after separation of the biomass (for example, after cell pellets are centrifuged and separated). In other embodiments, the PB hosting the recombinant vector is administered to a mammal, in particular a human, for in situ expression and delivery of the protein (use of the PB hosting said vector as a specific targeting tool for colon delivery of said protein). In this case, the extracellular medium is the ecosystem of the mammal, more particularly its intestine, and even more particularly its colon.

The recombinant vector according to the present invention comprises at least one suitable promoter for the expression of a protein in a PB. A “suitable promoter” can be constitutive and/or inducible; it is preferably inducible. Preferably, a promoter of propionic origin such as the promoter of protein PF963 of P. freudenreichii, used by the Inventors in the examples below, will be used. More preferably, strong propionic promoters are used. As noted by the Inventors, it could be advantageous to use a promoter ensuring “basic” constitutive expression which could be strengthened by induced expression under particular conditions (for example, conditions of digestive stress). For example, BCCP (biotin carboxyl carrier protein) is naturally very strongly expressed by P. freudenreichii in standard conditions, and even more so in the presence of bile or biliary salts (Hervé et al., 2007). Another example is the promoter for the Tuf protein, a major protein expressed by P. freudenreichii in standard conditions and even enhanced by digestive stresses (Leverrier et al., 2004; Falentin et al., 2010). The promoters for BCCP and Tuf are thus suitable promoters in the context of the invention. Advantageously, the vector according to the invention will contain at least two suitable promoters such as defined above which, even more advantageously, will be situated preferably in a series (but not necessarily coupled to each other), in order to increase the expression level of the protein (“expression booster” effect). For example, the promoters for proteins PF963 and BCCP, for proteins PF963 and Tuf, or for proteins BCCP and Tuf can be used at the same time.

The recombinant vector according to the present invention comprises at least one nucleotide sequence coding for a PB signal peptide for the secretion of a protein of interest. Preferably, a signal peptide of a PB selected from DPB and so-called “cutaneous” PB (CPB) will be used. Among other examples, the following DPB can be cited: Propionibacterium freudenreichii, P. jensenii, P. thoenii and P. acidipropionici. The following CPB can also be cited: P. acnes, P. granulosum, P. avidum and P. propionicum. Even more preferably, a signal peptide of a PB selected from the following species will be used: Propionibacterium freudenreichii, more particularly the subspecies P. freudenreichii subsp. freudenreichii and P. freudenreichii subsp. shermanii, and Propionibacterium acnes, wherein said PB is preferentially P. freudenreichii subsp. shermanii. More preferentially, a nucleotide sequence coding for a signal peptide will be selected from the following sequences:
sequences SEQ ID NO 1 to 18 (see table I below);
complementary sequences of same;
sequences at least 80% similar to same or to complementary sequences of same; and
sequences at least 80% hybridizable in strict conditions with same or with complementary sequences of same.

Table I below presents the nucleotide (NT) sequences mentioned above, as well as the corresponding amino acid (AA) sequences, identified following sequencing by the Inventors of the P. freudenreichii genome.

TABLE I

| Protein | | SEQ ID NO | |
|---|---|---|---|
| identified | Putative signal peptide | NT | AA |
| PF# 1058 | MSKTLSRIASVASVAALAGSITVIAGQNASA-DS | 1 | 19 |
| | Atgtcaaagacactctctcggatcgcatccgtcgcttcggttg | | |
| | Ccgcgctcgccggcagcatcaccgtcatcgccgggcagaa | | |
| | cgcgtccgccgacagc | | |
| PF# 1328 | MKNGLKTLLIGGVGIATLAVGGVGTAIA-DN | 2 | 20 |
| | Gtgaagaacggtctcaagaccctgctcattggtggagtcgg | | |
| | Catcgcgacccttgcggtcggcggcgtcggaactgccatcg | | |
| | cagacaat | | |
| PF# 1347 | MRSTTTKAFAGVAVLALALAGCGSNSGSSTKSA-DS | 3 | 21 |
| | Atgcgatccaccacgacgaaggcgtttgccggtgtcgctgtgctggcgct | | |
| | Ggcgcttgctggctgcggctcgaattcgggctcgtccaccaagtcggccg | | |
| | acagc | | |
| PF# 146 | MLTRKRVVAAGAAATLSLTAFAGLQPASA-AT | 4 | 22 |
| | Atgctcactcgcaagagagtggttgcagcgggagctgccgcc | | |
| | Accctgtccctcacggcgtttgccgggttgcagcccgccagcg | | |
| | ccgccacc | | |
| PF# 1885 | MGFRVGRRPLIGAVLAGSMATLVGCSTSGSGSGA-SS | 5 | 23 |
| | Atgggattcagggttggccgtcgtcccctcatcggggcagttctcgccgggt | | |
| | c | | |
| | Gatggcaacactcgtgggctgttccacctcgggtagcggcagtggagcctc | | |
| | cagc | | |
| PF# 190 | MQALQGRRRSRRVMAAAVAALTAMTVLPSQLNAVA-AP | 6 | 24 |
| | Atgcaggccctccaaggaaggcgccggtcacgacgggtgatggcggccgc | | |
| | Ggtagcagccctcaccgccatgaccgtgctgccctcccagctcaacgccgtt | | |
| | g | | |
| | ctgcaccc | | |
| PF# 2074 | MSTGRMKFIKLAVPVIVACCLTPMAALA-DV | 7 | 25 |
| | Atgtccactggccgcatgaagttcatcaagctggcagttcctg | | |
| | tcatcgttgcctgctgcttgacgccaatggctgccttagctgatgtg | | |
| PF# 241 | MAMRARHGVVRLGLVCLTALAVFGTANVSGQVAVMA-EG | 8 | 26 |
| | Atggcgatgagggcacgtcacggcgtcgtccggcttggtctggtctgtctca | | |
| | ccgc | | |
| | attggcggtcttcggcacggcaaatgtgtcgggtcaggttgcggtgatggct | | |
| | gagggc | | |
| PF# 2732 | MNQALSTMRLKIGDSTKRIRIFFVVMAVAITLLA-GR | 9 | 27 |
| | Ttgaaccaggccctgtcgacgatgcgcctgaagatcggcgactccacc | | |
| | Aagcgcatccggatcttcttcgtcgtgatggccgtggcgatcaccctgctc | | |
| | gcgggacgg | | |
| PF# 279 | MRRRTTIAALAAVLSFSPLAAQA-AP | 10 | 28 |
| | Atgcgacgtcgcaccacgattgcagccctcgctg | | |
| | Ctgtcttgagtttcagtcccctggccgcccaggccg | | |
| | caccc | | |
| PF# 2818 | MPSHAVRETRANKLRRFLRPTVAQGVLGIAFCLVAAVGVVQI-RS | 11 | 29 |
| | Atgcctagtcatgcggtgcgggagacgcgggcgaacaagttgcgccggttcc | | |
| | tgcggccc | | |
| | Accgttgcccagggcgtgctcggtatcgcgttctgcctcgtggccgccgtcg | | |
| | gcgtggtgca | | |
| | gatccgctcc | | |
| PF# 2932 | MSRIQLPRLSRIAIAAAASAALIGTSFIAPATAFA-AP | 12 | 30 |
| | Atgtcacggattcaactoccccggctgagccggattgcgatcgcagc | | |
| | Agcagcttccgctgccctgatcggcaccagcttcatcgccccggcca | | |
| | cggcctttgccgcgccg | | |
| PF# 3042 | MKRRTLLGTLGIMGLSVPLAACS-SK | 13 | 31 |
| | Ctagccgaccttctcggccttgctggccaggtcgtc | | |
| | ggggatcgtgacccccatcagggaggcggccttctcatt | | |
| PF# 3412 | MVTGGNDMPSKRITTWPGISALSALIAGMLLAPLPVAA-DG | 14 | 32 |
| | Ttagttgttggggacgaggagggagtggagttcgatgacgtcgagggtgggt | | |
| | gc | | |
| | Ggtggcggggcgggtgatggtttcggtgccggtgtgtccttgggcggtccag | | |
| | gtg | | |
| | atggtccaggt | | |
| PF# 3427 | MAMVMASLAMFGASRASA-AD | 15 | 33 |
| | Tcagccagttggtgccggccttggcgtcgg | | |
| | cggcgtgggatacacgcggaactgggcgcc | | |
| PF# 527 | MFISRFRRAAAVGLAAVTALSATACSGSSSSSSSSA-SS | 16 | 34 |
| | Atgttcatttcgcgcttccgtcgtgoggctgcggtcggcctggccgc | | |
| | Cgtcaccgcattgtccgccactgcctgtagcggttcctcgtcgtcgtc | | |
| | cagctcatccgcgagctcg | | |
| PF# 876 | MKSATRRPLTRWIVAFGVVLVLVIAGSVGLHASG-AL | 17 | 35 |
| | Atgaagtccgcgacgcgacgcccgctgacgcgctggattgtcgccttcg | | |
| | Gggtggtgttggtgctggtcatcgccgggtcggtggggctgcatgcctccg | | |
| | gtgccctg | | |
| PF# 963 | MNPFVKTARVAITSTLVAGSLATASLVFAPLAQA-DY | 18 | 36 |
| | Gtgaatcccttcgtcaagacggcgcgcgtggctatcacctcgacgc | | |
| | Tggtggcaggctcgctcgccactgccagcctcgtgtttgcaccactt | | |
| | gcacaggccgattac | | |

Even more preferably, a nucleotide sequence coding for a signal peptide will be selected from the following sequences:
sequence SEQ ID NO 18 coding for the signal protein of protein PF963 of P. freudenreichii (table I above);
the complementary sequence of same;
sequences at least 80% similar to same or to the complementary sequence of same; and
sequences at least 80% hybridizable in strict conditions with same or with the complementary sequence of same.

A nucleotide sequence that is “complementary” to a reference nucleotide sequence refers herein to any DNA whose nucleotides are complementary to those of the reference sequence, and whose orientation is reversed (the complementary sequence is thus an antiparallel sequence). Two “complementary” nucleotide sequences are thus such that each base of one is paired with the complementary base of the other, with the orientation of the two sequences being reversed. The complementary bases are A and T (or A and U in the case of RNA), and C and G.

A nucleotide sequence that is “similar” or “homologous” to a reference nucleotide sequence refers herein to a nucleotide sequence with a percent identity with the reference nucleotide sequence of at least roughly 80%, preferably at least roughly 85%, more preferably at least roughly 90%, even more preferably at least roughly 95%, even more preferably at least roughly 98%, wherein said percentage is purely statistical and the differences between the two nucleotide sequences can be distributed randomly over their entire length. Similar sequences can thus include variations related to mutations in the reference sequence, wherein said mutations correspond in particular to truncations, substitutions, deletions and/or additions of at least one nucleotide. Similar sequences can also include variations related to degeneration of the genetic code.

In particular, sequences “at least roughly X % similar” to a reference nucleotide sequence coding for a PB signal peptide refer to variants of this sequence, wherein said variants have, over their entire length, at least roughly X % of bases identical to those of the reference sequence. The identical bases can be consecutive in their entirety or only in part. The variants thus envisaged can be the same length as the reference nucleotide sequence, or a different length, as it acts as a signal peptide in a PB. Indeed, those persons skilled in the art know that the expression products of nucleotide sequences with a certain level of similarity (at least roughly X %) can nevertheless, taking into account degeneration of the genetic code on the one hand, and the preferential use of certain codons according to the host organisms (bacteria, yeasts, etc.) on the other, fulfill the same function. These expression products can themselves be identical or similar. Here, these expression products are functional signal peptides in PB.

The definition above can be extended to amino acids sequences, or peptide/protein sequences, at least roughly X % similar to a reference amino acid sequence. This case includes protein variants with, over their entire length, amino acids at least roughly X % similar to those of the reference sequence. Here again, the similar amino acids can be consecutive in their entirety or only in part. Peptide/protein variants can be the same length or a different length, given that, preferably, the biological function of the reference amino acid sequence is conserved.

The expression “similar amino acids” refers herein to amino acids with the same side-chain reactivity. Thus, polarity and comparable ionization properties are used by persons skilled in the art to define groups of similar amino acids. For example, it is useful to categorize aliphatic amino acids, namely glycine, alanine, valine, leucine and isoleucine, within the same group. Similarly, dicarboxylic amino acids, aspartic acid and glutamic acid are similar. Also, serine and threonine belong to the same group in that they both carry an esterifiable alcohol group. Additionally, lysine, arginine and histidine can be cited as similar basic amino acids, etc.

In all the definitions above, “X” equals 80. In particular, “X” equals 85, preferably 90, more preferably 95 and even more preferably 98.

A sequence that is “hybridizable in strict conditions” with a reference nucleotide sequence refers herein to a nucleotide sequence capable of hybridizing under temperature and ionic strength conditions suitable to maintain hybridization between two complementary fragments of DNA. The “strict hybridization conditions” are consistent with the classic definition known to those persons skilled in the art (Sambrook and Russell, 2001). “Strict hybridization conditions” are, for example, conditions that enable the specific hybridization of two single-stranded nucleotide sequences after at least one washing step as described below. The hybridization step can in particular be carried out at roughly 65° C. for 12 h in a solution comprising 6×SSC, 0.5% SDS, 5×Denhardt's solution and 100 μg nonspecific DNA (salmon sperm DNA, for example), or in any another solution of equivalent ionic strength. The following step, comprising at least one washing, is carried out, for example, at roughly 65° C. in a solution comprising at most 0.2×SSC and at most 0.1% SDS, or in any another solution of equivalent ionic strength. The parameters defining the hybridization conditions depend on the temperature (Tm) at which 50% of the paired strands separate. For sequences of more than 30 bases, the temperature (Tm) is calculated according to the formula: Tm=81.5+0.41*[% G+C]+16.6*Log(cation concentration)−0.63*[% formamide]−(600/number of bases). For sequences of less than 30 bases, the temperature (Tm) is defined by the following relationship: Tm=4*(number of G+C)+2*(number of A+T). The hybridization conditions can thus be adapted by those persons skilled in the art according to the size of the sequences used, their GC content and other parameters, as indicated in particular in the protocols described in Sambrook and Russell (2001).

In particular, “reference nucleotide sequences” and “reference amino acid sequences” from P. freudenreichii can be obtained from its genomic sequence that has been recently made available by the Inventors (Falentin et al., 2010, Plos One; Genbank accession No. FN806773).

Preferably, a signal peptide will be selected from:
sequences SEQ ID NO 19 to 36 (table I above) and 45 to 57 (see table II below);
sequences at least 80% similar to same; and
analogs and derivatives of same.

More preferably, said signal peptide will be selected from:
sequence SEQ ID NO 36 corresponding to the signal peptide of protein PF963 of P. freudenreichii (table I above);
sequences at least 80% similar to same; and
analogs and derivatives of same.

Table II below presents said amino acid sequences, identified by the Inventors from the genomic sequence of P. acnes, accessible from databases (strain P. acnes KPA171202; accession number: NCBI: NC_{—}006085; GenBank: AEO17283).

TABLE II

| Protein | | | | |
|---|---|---|---|---|
| sequence | Putative | SEQ | | Homologous |
| accession | sequence of the | ID | Associated | protein in P. |
| number | signal peptide | NO | function | freudenreichii |
| PPA2239 | MSKVVASAIA | 45 | | PF1328 |
| | GALSTLSAGG LTMVQA | | | |
| PPA1840 | mrkaivtpva vlavlvmalt | 46 | | PF1347 |
| | gcgqknqsgg | | | |
| PPA1786 | mastprrrwa wvlllvvasl | 47 | | PF1885 |
| | vivgvyrka | | | |
| PPA2198 | mssmkglslv latsfmlsfs | 48 | | PF2074 |
| | pgssfa | | | |
| PPA0721 | mehrygasqv sgsaprrgrg | 49 | | PF241 |
| PPA2198 | mssmkglslv latsfmlsfs | 50 | | PF3412 |
| | pgssfas | | | |
| PPA0257 | mphsdqptsk rvmsaprrrm | 51 | | PF876 |
| | pgwvpvtvgi avvvivvvav | | | |
| | ivsslrs | | | |
| AAA51650 | mfgtpsrrtf ltasalsama | 52 | hyaluronidase | |
| | laasptvtda | | | |
| | ia | | | |
| CAA67627 | mkinarfavm aasvavlmaa | 53 | triacylglycerol | |
| | apiaqa | | lipase | |
| AAT83976 | mypvhlplrn esefsfrahn | 54 | lipoprotein | |
| | hggtvpsrlt rrsvlatgav | | | |
| | alpmtaaaca | | | |
| AAT83859 | mrhmrplial slaglmtlsa | 55 | peptide-binding | |
| | cgedvaa | | protein | |
| AAT83771 | mnrtlkvaav gaiailclaa | 56 | secreted sugar- | |
| | csdpgsdsaq s | | binding protein | |
| AAT83059 | mekssfaaan mtimsepttp | 57 | secreted | |
| | tsqa | | protease | |

A particularly preferred recombinant vector is obtained by inserting a nucleotide sequence coding for a protein of interest in vector pFB4 (contained in the recombinant strain Escherichia coli DH5α deposited with the Collection Nationale de Cultures de Microorganismes (CNCM, Institut Pasteur, 25 rue du Docteur Roux, 75724 Paris Cedex 15, France) on Apr. 15, 2010 and registered under number I-4297).

In the recombinant vector according to the invention, the PB signal peptide is “fused translationally” with the protein of interest, an essential condition so that it can be secreted by the host cell.

It is observed that a signal peptide could be translationally fused with several amino acid sequences in a series, making it possible to express and secrete a chimeric protein, for example. Alternately or additionally, the same vector can possibly carry several translational fusions of a signal peptide and a protein of interest, under the control of one or more promoters. The promoters in question can control the transcription of only one or of several of these translational fusions.

In the context of the present invention, the amino acid sequences of interest (or proteins of interest) that will be expressed from the recombinant vector of the invention are eukaryotic. They are preferably from animal origin, more preferably from mammalian origin. In particular, they can originate from mammals selected from rodents such as mice, rats, rabbits, Chinese pigs, hamsters; canidae (e.g., dogs) and felidae (e.g., cats); domestic livestoc, including cows, pigs, goats, sheeps, horses; and humans. Even more preferably, the amino acid sequences of interest are from human origin.

Preferably, at least one eukaryotic amino acid sequence of interest to be expressed and secreted using the recombinant vector according to the present invention has a chemical mediation activity.

Advantageously, any or all of the eukaryotic amino acid sequences of interest to be expressed and secreted using the recombinant vector according to the present invention has(ve) a chemical mediation activity.

In other words, at least one of the eukaryotic amino acid sequences of interest (or proteins of interest) that will be expressed from the recombinant vector according to the present invention has a biological activity of interest, preferably a chemical mediation activity.

The terms and expressions “activity,” “function,” “biological activity,” “biological function,” “bioactivity,” “(biological) activity of interest” and “(biological) function of interest” are equivalent and refer to a biological activity that is of interest, especially for medical purposes, such as an activity of chemical mediation. In particular, the protein that will be expressed from the vector according to the present invention is “functional” or “active” or “bioactive,” that is, it is able to fulfill its natural biological function, which is independent (from a qualitative and/or quantitative point of view) of post-translational modifications not able to be carried out by a PB.

Any eukaryotic protein whose biological activity is of interest to industry or medicine is thus within the scope of the present invention, insofar as said activity does not depend on post-translational modifications not able to be carried out by a PB.

The principal utility of the invention is to make it possible to produce an eukaryotic protein of interest. It is described here another utility of the invention that is to recycle industrial organic waste or residual by-products. Thus, for example, whey and molasses, which are produced as unused residues by certain industries, may be recycled as substrates for the culture of recombinant PB able to synthesize proteins of interest.

An eukaryotic protein, or a fragment or domain thereof, having an activity of “chemical mediation” is a “chemical mediator”, i.e., a protein naturally secreted by an eukaryotic cell, or a fragment or domain of such a protein, and that is capable of binding a cell receptor to induce a cellular response. Examples of chemical mediators include neuromediators or neurotransmitters, hormones, growth factors, cytokines, and the like. Chemical mediators also include fusion proteins capable of binding to a cell receptor.

Preferably, the eukaryotic protein of interest that is expressed and secreted from the vector according to the invention, has a chemical mediation activity that is of medical interest such as an activity selected from proapoptotic, anti-inflammatory, immunomodulatory activities, and combinations thereof. The protein of interest may be selected from cytokines, chemokines, peptide hormones, neurotransmitters, peptides involved in inflammation, satiety, blood pressure, etc. The protein of interest is preferably a proapoptotic and/or anti-inflammatory protein, preferably from human origin. According to a preferred embodiment, the protein of interest is a cytokine.

The amino acid sequence of interest can be that of a protein or of a biologically active fragment or domain thereof. This means that when the amino acid sequence that is expressed from the vector according to the present invention is a protein fragment or domain, it remains “functional” or “active” or “bioactive,” that is, it is able to fulfill the natural biological function of the corresponding native protein. All definitions provided herein with respect to proteins also apply to biologically active fragments or domains thereof.

In a preferred embodiment, the recombinant vector according to the present invention makes it possible to express and secrete the proapoptotic protein TRAIL (TNF-related apoptosis-inducing ligand, also called TNSF10, TL2, CD253 and Apo-2L), a cytokine of the TNF family. In particular, the amino acid sequence of interest is that of the active C-terminal extracellular domain of TRAIL, preferably the sequence from amino acids 114 to 281 of TRAIL (TRAIL sequence accession number in GenBank: U37518; Uniparc: UPI0000001629).

According to the literature, TRAIL is an antineoplastic agent with strong potential because it induces the death of many tumor cells, independently of p53 and Pgp180 (MDR, multidrug resistance). TRAIL also inhibits the growth of xenografted colon tumors in nude mice (Ashkenazi et al., 1999). Quite interestingly, TRAIL has little cytotoxic effect on most normal tissues (Ashkenazi et al., 1999), including human colon epithelium (Sträter et al., 2002).

Other teams very recently expressed the active C-terminal extracellular domain of TRAIL in bacteria such as Salmonella typhimurium (Ganai et al., 2009), Bifidobacterium longum (Hu et al., 2009) and E. coli (Zhang et al., 2010). In this work, salmonellas, bifidobacteria and coliform bacteria are proposed as systemic TRAIL delivery vectors in cancer models.

However, PB have major advantages compared to other bacteria such as salmonellas, bifidobacteria and coliform bacteria.

First, DPB enable local delivery because they target colon epithelial cells and have an active fermentative metabolism in the human colon (Hervé et al., 2007), which enables site-specific delivery of TRAIL.

Second, DPB themselves have proapoptotic properties. It was recently shown in vitro that DPB, by the SCFA resulting from their fermentative metabolism, induce the apoptosis of two human colon adenocarcinoma cell lines (Caco2 and HT29) (Jan et al., 2002). This effect is directly related to the release of propionate by these bacteria and to their action on cancer cell mitochondria. It has also been shown that at extracellular pH (pHₑ) between 6 and 7.5, SCFA (propionate and acetate SCFA) induce apoptotic death whereas at pHₑ=5.5, they induce necrotic death in HT29 human colon cancer cells (Lan et al., Apoptosis, 2007). In vivo, these food-quality (GRAS: generally regarded as safe) bacteria adapt and survive in the digestive tract of animals and humans with an efficiency that, although strain-dependent, exceeds that of other probiotics (Hervé et al., 2007). Moreover, they express in the intestine enzymatic activities characteristic of their fermentative metabolism by producing an increase in SCFA concentrations (Lan at al., Br. J. Nutr., 2007) and induce an increase in apoptosis in the mucosa of the colon of rats treated with 1,2-dimethylhydrazine (Lan et al., 2008). The Inventors have further shown a synergistic action with TRAIL in vitro as illustrated in the examples below.

A particularly preferred recombinant vector is the vector pFB4:TRAIL, wherein the amino acid sequence of interest is the sequence from amino acids 114 to 281 of the TRAIL C-terminal extracellular domain. This vector is hosted by type strain CIP103027 of P. freudenreichii subsp. shermanii, deposited on Jul. 23, 2009 under number I-4213 with the Collection Nationale de Cultures de Microorganismes (CNCM, Institut Pasteur, 25 rue du Docteur Roux, 75724 Paris Cedex 15, France).

The present invention further relates to a recombinant propionibacterium comprising at least one recombinant vector as previously defined.

Advantageously, the recombinant vector will be carried by the chromosome of the bacterium according to the invention. The vector can be integrated, for example, in the chromosome of the host cell by homologous recombination.

One particularly preferred bacterium is type strain CIP103027 of P. freudenreichii subsp. shermanii, deposited with the CNCM on Jul. 23, 2009 under number I-4213.

If the protein to be secreted lends itself to such a use (an anorexiant peptide, for example), the bacterium according to the invention could be used as a probiotic food or dietary supplement for mammals, in particular humans. Advantageously, the bacterium will be integrated in the mammal's food in the form of a fermented dairy product (e.g., fermented milk, fermented whey, cheese).

The present invention further relates to a recombinant vector or a recombinant propionibacterium as defined above, for the use of same as a drug.

The present invention further relates to a drug (or pharmaceutical composition) comprising an effective quantity of at least:
one vector according to the invention; and/or
one bacterium according to the invention,
and at least one pharmaceutically acceptable carrier (or excipient).

In said drug, the vector and/or the bacterium are advantageously used as therapeutic agents. A drug according to the invention can be manufactured in a conventional way. A drug in accordance with the invention can moreover include one or more pharmaceutically acceptable excipients or additives such as diluents, adjuvants, anti-foaming agents, stabilizers, dispersants, colorants, preservatives, etc. Inert excipients or adjuvants can be used in such a way that, in the drugs according to the present invention, the only therapeutic agents will be the vector and/or the bacterium. Nevertheless, the drug according to the present invention can include one or more other therapeutically or prophylactically active agents, in addition to the vector and/or the bacterium. Advantageously, the combination of several therapeutic agents, including at least the vector and/or the bacterium, will have a better therapeutic or prophylactic action than when the vector and/or the bacterium are the only therapeutic agents present in the drug. This better action can be, among others:
a better dose-effect relationship;
a therapeutic or prophylactic effect that is more stable or longer lasting over time;
a better administration of the drug;
a synergy of action between at least two therapeutic agents present in the drug.

Preferably, a drug in accordance with the present invention is intended to prevent and/or to treat at least one disease selected from allergies, hypertension (e.g., the protein of interest has a hypotensive activity), obesity (e.g., the protein of interest has an anorexiant activity), colorectal cancers (e.g., the protein of interest has a proapoptotic activity) and inflammatory colon diseases, in particular Crohn's disease (wherein the protein of interest is advantageously an anti-inflammatory cytokine, for example IL-10), etc. Alternately, a drug of the invention is intended to prevent at least one microbial infection, for example a viral, bacterial, fungal or parasitic infection, etc. In this case, the protein of interest will be an antigen or an epitope, for example. Thus, said drug advantageously will be a vaccine, in which case the pharmaceutically acceptable carrier could be an immune adjuvant.

The various means of the present invention (vector, bacterium, drug) as described above are preferably administered to a mammal for the secretion of the proteins of interest in the small intestine and/or the colon, preferably the colon, of said mammal.

The term “mammal” is defined in its usual sense. Examples of mammals include bovines; pigs; goats; sheep; horses; rodents such as mice, rats and hamsters; felines and canines, including domestic animals such as cats and dogs. A preferred mammal in the context of the invention is a human.

The means of the invention (vector, bacterium, drug) can be administered by any suitable conventional route, in particular selected from the oral, subcutaneous, intramuscular, intravenous and intratracheal routes. Oral administration is preferred, wherein the drug is in the form of tablets, hard gelatin capsules (e.g., gastroprotective gelatin capsules), soft capsules, powders for direct use or for dilution (e.g., lyophilisates), syrups, gels, etc. Said means can be administered in a single or repeated dose one or more times spaced over a certain interval of time. The suitable administration route and dosing schedule can vary according to various parameters, such as the subject to be treated and/or the protein of interest.

The invention further relates to a method of therapeutic or prophylactic treatment, wherein a therapeutically effective quantity of a vector and/or a bacterium and/or a drug according to the invention is administered to a subject in need of such a treatment.

The present invention further relates to the use of a vector and/or a bacterium in accordance with the preceding description to produce and secrete, preferably in the extracellular medium, one or more proteins of interest.

The present invention further relates to a method for producing and secreting in the extracellular medium, by a bacterium according to the invention, at least one amino acid sequence of interest as defined above, wherein said method comprises at least:
culturing said bacterium under suitable conditions;
recovering the culture medium containing said amino acid sequence of interest (since it is secreted in the culture medium by the bacterium); and
optionally, purifying said amino acid sequence of interest.

Preferably, the method according to the invention makes it possible to produce and secrete proteins on a large scale, that is, on an industrial scale (in protein production plants).

The “suitable conditions” (in terms of the composition of the culture medium, temperature, time, ventilation, stirring, etc.) for culturing PB are known to those persons skilled in the art (see in particular documents US 20090312425 in the name of Meiji Dairies Corp. and CN 101045910 in the name of Nanjing University of Technology). Once again, these bacteria are robust, are able to grow on particular substrates such as whey or molasses and are able to adapt to non-standard culture conditions, characteristics which a large number of other bacteria do not share.

Protein purification calls upon the general knowledge of those persons skilled in the art and can be carried out without any difficulty using classic techniques.

The invention further relates to pharmaceutical products containing at least one PB, preferably one non-recombinant PB, and at least one protein of interest as a combination product for prophylactic or therapeutic use, in mammals, that is simultaneous, separated or sequential over time.

The pharmaceutical products according to the invention can comprise the protein itself or a nucleotide sequence coding for said protein, wherein said nucleotide sequence is optionally carried by a suitable expression vector.

For example, the pharmaceutical products in accordance with the present invention are intended to prevent and/or to treat at least one disease selected from allergies, hypertension (e.g., the protein of interest has a hypotensive activity), obesity (e.g., the protein of interest has an anorexiant activity), colorectal cancers (e.g., the protein of interest has a proapoptotic activity) and inflammatory colon diseases, in particular Crohn's disease (wherein the protein of interest is advantageously an anti-inflammatory cytokine, for example IL-10), etc. Alternately, said products can be intended to prevent at least one microbial infection, for example a viral, bacterial, fungal or parasitic infection, etc. In this case, the protein of interest will be an antigen or an epitope, for example.

Advantageously, for an antineoplastic therapy, in particular for the treatment of colorectal cancers, the pharmaceutical products according to the present invention preferably comprise:
at least one non-recombinant PB such as P. freudenreichii subsp. shermanii, and
at least the TRAIL protein or the sequence from amino acids 114 to 281 of the TRAIL C-terminal extracellular domain.

In said pharmaceutical products, the PB can be administered in the form of a probiotic which will be added to the mammal's food and which will serve, for example, as an adjuvant TRAIL-based chemotherapy. In the latter case, it is recalled that, as being typically administered systemically, conventional chemotherapeutic treatments have a lot of undesirable side-effects, in particular due to a high dosage regime and a lack of specificity. Thus, by orally administering the probiotic BP as an adjuvant to a TRAIL-based chemotherapy, the bacteria and TRAIL will be able to exhibit an enhanced, advantageously synergistic, anti-tumoral activity in the colon, resulting in noticeably reduced (or even prevented) side-effects by reducing the required dosages and increasing the specificity of the treatment.

Alternatively, still in reference to an antineoplastic therapy, in particular for the treatment of colorectal cancers, the pharmaceutical products according to the present invention comprise:
a culture supernatant of a non-recombinant PB such as P. freudenreichii subsp. shermanii, wherein said supernatant has optionally undergone one or more suitable conventional treatments to improve its harmlessness and/or preservation and/or physicochemical properties etc., and
at least the TRAIL protein or the sequence from amino acids 114 to 281 of the TRAIL C-terminal extracellular domain.

As the examples illustrate below, the Inventors indeed have shown a synergy of proapoptotic action on HT29 colon cancer cells between PB and/or PB culture supernatants (containing SOFA, in particular propionate and/or acetate SCFA) and TRAIL.

Alternately again, and still in reference to an antineoplastic therapy, in particular for the treatment of colorectal cancers, the pharmaceutical products according to the present invention comprise:
one or more SCFA, in particular propionate and/or acetate SOFA, advantageously obtained from the culture supernatant of one or more non-recombinant PB such as P. freudenreichii subsp. shermanii, and
at least the TRAIL protein or the sequence from amino acids 114 to 281 of the TRAIL C-terminal extracellular domain.

The invention further concerns a method for treating a cancer in a mammal in need thereof, comprising administering to said mammal:
at least one short-chain fatty acid (SOFA), preferably propionate and/or acetate, advantageously obtained by fermentation of at least one Propionibacterium such as Propionibacterium freudenreichii, and
proapoptotic TNF-related apoptosis inducing ligand (TRAIL/Apo-2 ligand), or a functional fragment thereof.

Said mammal is as defined above.

Said cancer is preferably a colorectal cancer.

Said functional fragment of TRAIL preferably comprises amino acid sequence from position 114 to position 281 of the TRAIL C-terminal extracellular domain.

Said SCFA and said TRAIL or functional fragment thereof may be administered to said mammal simultaneously, separately or sequentially.

The present invention is illustrated by the following figures:

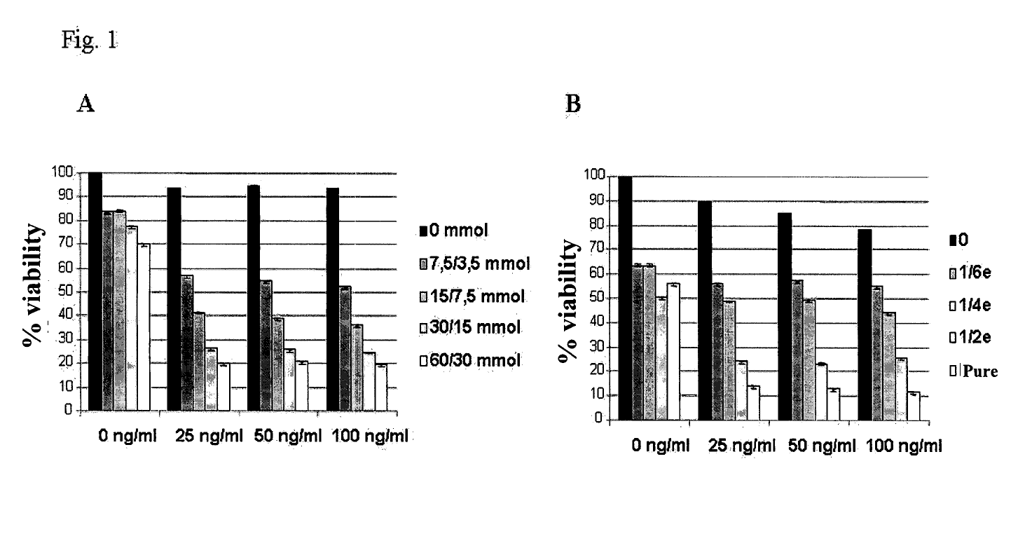

FIG. 1: Graphs illustrating the synergy observed in vitro between TRAIL and P. freudenreichii metabolites. HT29 colon cancer cells were treated with sublethal doses of TRAIL (25 ng/ml, 50 ng/ml and 100 ng/ml). Various doses of SCFA (propionate/acetate SCFA, FIG. 1A) or of P. freudenreichii supernatant (FIG. 1B) were used in co-treatment. Viability of the HT29 cells was determined after 24 hours of treatment.

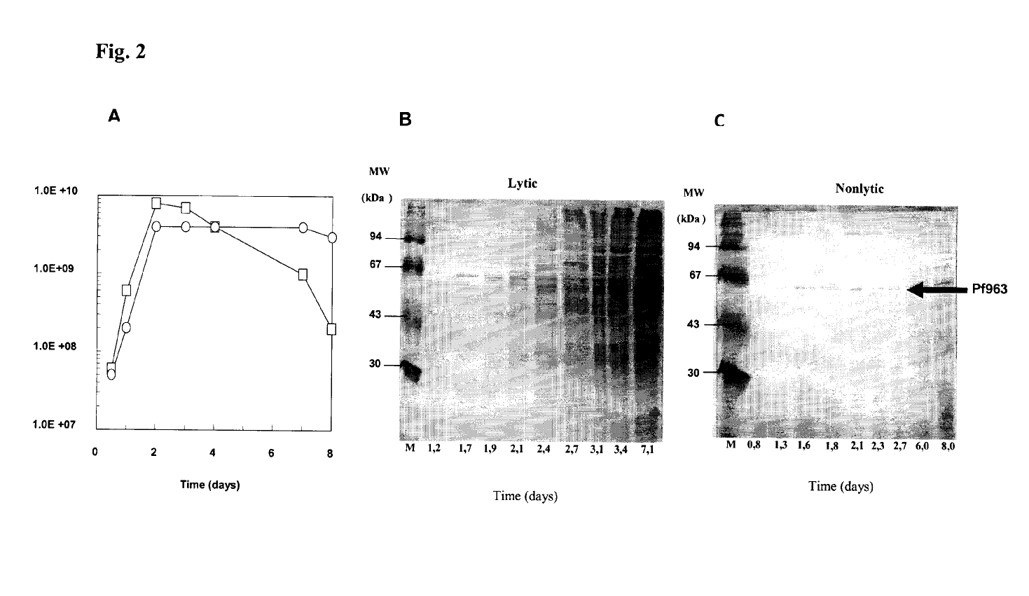

FIG. 2: Identification of the protein secreted in the majority by P. freudenreichii, PF963. A: growth of two strains of P. freudenreichii, one autolytic (□) and the other nonlytic (∘). B and C: electrophoretic analysis (SDS-PAGE) of proteins secreted by a strain of lytic (B) and nonlytic (C)P. freudenreichii. Protein PF963 was identified by mass spectrometry.

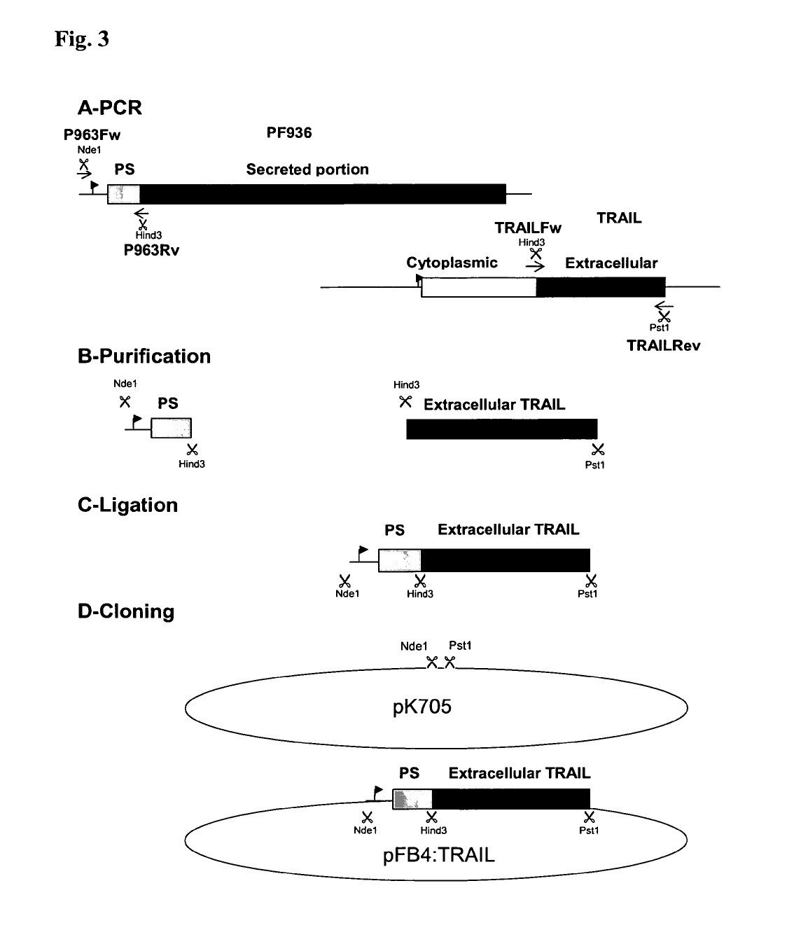

FIG. 3: Diagram detailing the cloning strategy to obtain the pFB4:TRAIL plasmid (deposited with the CNCM on Jul. 23, 2009 under number I-4213). A: the promoter region and signal peptide of the P. freudenreichii protein PF963 were amplified by PCR with introduction (PCR mutagenesis) of restriction sites Nde1 and HindIII. The active extracellular portion of TRAIL (residues 114 to 281) was amplified by PCR with introduction of restriction sites HindIII and Pst1. B and C: the two PCR products were purified and linked in order to obtain the ligation product SP-TRAIL. D: plasmid pK705 was opened by digestion using two enzymes Nde1 and Pst1. The ligation product SP-TRAIL was introduced into the open plasmid. The new plasmid pFB4 includes a promoter and a signal peptide enabling the secretion by P. freudenreichii of a heterologous protein. The arrows and the scissors represent PCR primers and restriction sites, respectively.

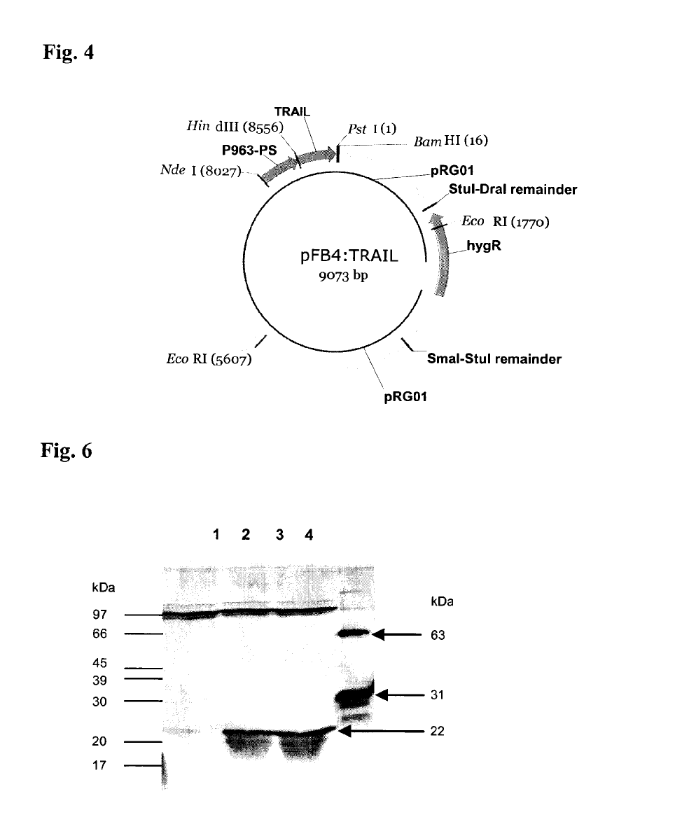

FIG. 4: Map of the pFB4:TRAIL plasmid (deposited with the CNCM on Aug. 13, 2009 under number I-4213).

FIG. 5: Sequence of the fusion protein coded for by the pFB4:TRAIL plasmid. In this sequence, the underlined region corresponds to the PF963 protein signal sequence and the region in bold corresponds to the TRAIL extracellular domain sequence (residues 114 to 281).

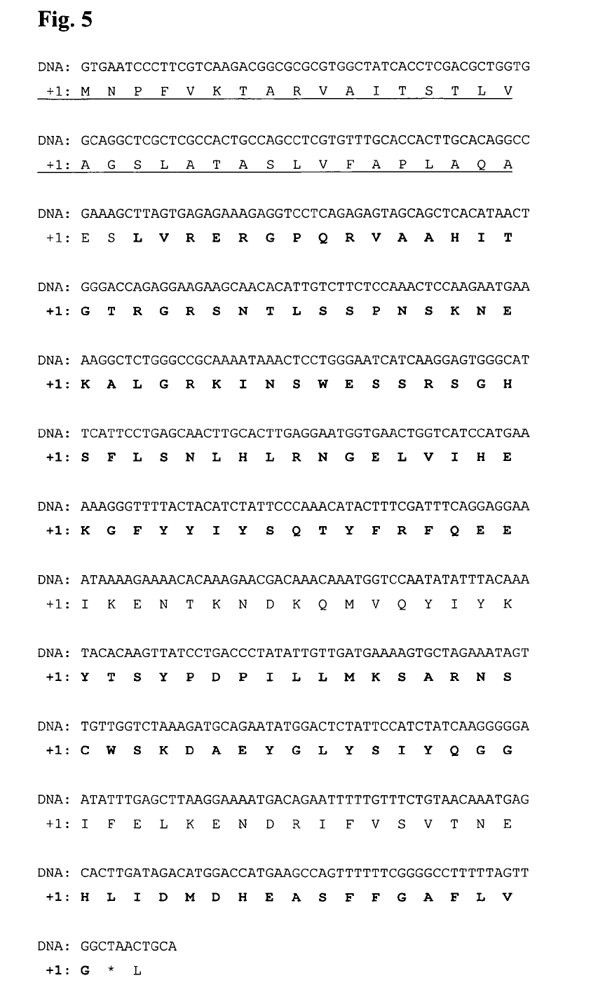

FIG. 6: Detection by Western blot of the fusion protein coded for by the pFB4:TRAIL plasmid. The samples deposited were culture supernatants of wild P. freudenreichii CIP103027 (1) or of P. freudenreichii CIP103027 carrying the pFB4:TRAIL plasmid (2 and 3). A solution of SuperKillerTRAIL™ (Alexis Biochemicals, Coger, France) was deposited as positive control (4). The Western blot was developed using a commercial “PAb to TRAIL” antibody (Alexis Biochemicals).

The following non-limiting examples, which refer to the figures above, illustrate the embodiments and advantages of the present invention.

### EXAMPLES

### I—Induction of the Intrinsic Mitochondrial Pathway of Apoptosis by Propionibacterium freudenreichii

During preliminary studies, the Inventors showed that certain selected strains of P. freudenreichii surviving the stresses undergone during intestinal transit in humans (Hervé et al., 2007), as well as in the rat (Lan et al., 2007), express the genes coding for fermentative metabolism enzymes and produce propionate and acetate short-chain fatty acids (SCFA) in situ in the colon (Lan et al., 2007).

Furthermore, this bacterium induced the apoptosis of human colon adenocarcinoma cells in vitro via these SCFA which act on cancer cell mitochondria (Jan et al., 2002). The mitochondrial pathway of apoptosis induction has been clearly identified in the triggering of programmed cell death of HT29 cells by dairy propionibacteria (Jan et al., 2002; Lan et al., 2007). Said SCFA cause the opening of mitochondrial permeability transition pores (PTP), the depolarization of mitochondria, the leaking of proapoptotic mitochondrial proteins and the activation of effector caspases.

Such an induction of apoptosis was then researched in vivo in a rat model of human digestive flora. Rats were treated or not treated by the carcinogen dimethylhydrazine (DMH) for the purpose of causing the appearance of damaged colonic epithelial cells likely to develop into colon cancer. These rats received by gavage, or did not receive, the P. freudenreichii bacterium. Colonic epithelial cell apoptosis and proliferation were quantified by anatomopathological analysis of histological sections of colon. The administration in healthy rats of P. freudenreichii had no effect on these parameters. On the other hand, a significant increase in apoptosis was observed in the rats treated with DMH (Lan et al., 2008). It thus appears that a specific apoptosis of cancer cells can be induced by dairy propionibacteria.

### II—Induction of the Extrinsic Pathway of Apoptosis by TRAIL Via Death Receptors, Synergy with Propionibacterium freudenreichii

TRAIL is a cytokine capable of inducing the apoptosis of human colon cancer cells by binding to death receptors. TRAIL thus induces a different apoptotic pathway on the cellular and molecular levels and potentiates the action of other proapoptotic molecules used in cancer chemotherapy (Lacour et al., 2001; Lacour et al., 2003; Meurette et al., 2005; Meurette et al., 2006). Cell death induced by TRAIL or by SOFA is promoted by an acidic environment (Meurette et al., 2007; Lan et al., 2007).

By viability tests (FIG. 1), and by in vitro apoptosis quantification methods (Hoechst staining and caspases activity, data not shown), the Inventors showed a synergistic proapoptotic effect of the cytokine TRAIL in combination with the propionate/acetate mixture or the propionibacteria culture supernatant in HT29 human colon cancer cells (FIG. 1). More precisely, the viability illustrated in FIG. 1 was determined using the following cytotoxicity test. HT29 human colon cancer cells (ATCC, Biovalley) were cultured in 96-well plates (30,000 cells/well) for 24 hours. They were then treated with TRAIL (0 ng/ml, 25 ng/ml, 50 ng/ml and 100 ng/ml) (SuperKillerTRAIL™, Alexis Biochemicals, Coger, France) in the presence of increasing concentrations of propionate/acetate (7.5 mmol/3.5 mmol; 15 mmol/7.5 mmol; 30 mmol/15 mmol; 60 mmol/30 mmol) or of bacterial supernatant (P. freudenreichii bacteria) (⅙, ¼, ½, pure). At the end of treatment (24 hours), the medium was discarded and the adherent cells were washed three times with 1×PBS (100 μl/well) and fixed in 99% ethanol (100 μl/well) for 30 minutes. After discarding the ethanol, the fixed cells were air dried and then stained for 30 minutes with methylene blue (diluted in 1× borate buffer). After three washings in water and drying (roughly 30 minutes), 100 μl of hydrochloric acid (0.1 N) was added to the wells. The plates were then analyzed by spectrophotometer at a wavelength of 620 nm (iEMS Reader MF; Lab-systems, Helsinki, Finland).

FIG. 1 shows that sublethal doses of TRAIL (25 ng/ml, 50 ng/ml and 100 ng/ml) do not significantly induce cell death during the treatment period. Moreover, the smallest doses of SCFA alone induce little or no cell death, but induce massive death in the presence of TRAIL (FIGS. 1A and 1B). These results show a synergy of proapoptotic action on human colon cancer cells between SCFA metabolites produced by PB and TRAIL.

### III—Development of a Recombinant Propionibacterium with the Goal of Inducing Both the Intrinsic and Extrinsic Apoptotic Pathways

3-1 Summary

The Inventors sought to make a bacterium, harmless to healthy cells, produce inducers of the two apoptotic pathways. These inducers are the SOFA produced by P. freudenreichii for the intrinsic pathway and TRAIL for the extrinsic pathway. Since propionibacteria have a positive tropism for the mucosa of the colon, said recombinant bacterium will not only be likely to produce TRAIL in situ in the colon, but also to carry SOFA and TRAIL toward colon epithelial cells.

To this end, a recombinant propionibacterium expressing TRAIL fused with a secretion signal peptide was developed for in situ production in experimental models of cancer colon.

Briefly, the major protein secreted by P. freudenreichii during its growth and in the absence of lysis, named PF963, was identified. The experimental procedure (e.g., electrophoresis, trypsinolysis, nano-LC and MS/MS) which led to the identification of PF963 is similar to that which had previously enabled the Inventors to identify GAPDH (Tarze et al., 2007). Very briefly, the supernatant of the nonlytic strain of P. freudenreichii was analyzed by electrophoresis. The gel fragment containing the major protein secreted was removed, rinsed and then subjected to “in gel” trypsin proteolysis. The resulting peptides were separated by nano-LC and then analyzed with tandem mass spectrometry (MS/MS).

PF963 is an enzyme secreted via the machinery of the “Sec” pathway which recognizes and cleaves a signal peptide (SP). By genetic engineering, said SP was fused with the active C-terminal extracellular domain of TRAIL. This construction was carried out in E. coli on a cloning plasmid. The fusion thus obtained was introduced into an expression vector (pK705) previously developed for the cloning and expression of propionibacterial genes in dairy propionibacteria and efficient in P. freudenreichii (Kiatpapan et al., 2000) in order to express the fusion protein. The expression and the extracellular addressing of the fusion protein were then analyzed by Western blot.

According to FIG. 2A, the growth of P. freudenreichii shows that certain strains are lysed (□) and others not (∘). In the latter case, protein PF963 is secreted in the medium (FIG. 2C) without leakage of cytoplasmic proteins as in the case of spontaneous bacterial lysis (FIG. 2B). The upstream portion of the PF963 gene, comprising the promoter and the signal peptide, was amplified by PCR and fused with the C-terminal portion of TRAIL (FIGS. 3A to 3C). The following step consisted of its introduction in a P. freudenreichii expression plasmid (FIG. 3D).

3-2 Obtaining the Strain Propionibacterium freudenreichii subsp. shermanii CIP103027 (TL34) Carrying the pE134:TRAIL Plasmid.

3-2-1 Identification of the Protein PF963 Secreted by Propionibacterium freudenreichii subsp. shermanii.

In order to identify a secreted protein, strains were screened on the basis of aptitude for autolysis. Indeed, it is known that certain strains of said bacterium make use of a programmed cell suicide, autolysis. In this case, cytoplasmic proteins are released in the surrounding medium. On the other hand, other strains, including strain CIP103027, do not undergo autolysis and on the contrary make use of a tolerance reaction with respect to various stresses, called the starvation-induced multi-tolerance response. In principle, these nonlytic strains thus only release actively secreted proteins and do not release proteins by accident. FIG. 2A shows the evolution of the bacterial population for an autolytic strain (□) and for a nonlytic strain (∘) of Propionibacterium freudenreichii subsp. shermanii. FIG. 2C shows the electrophoretic analysis (SDS-PAGE) of proteins secreted by a nonlytic strain, CIP103027. This analysis reveals several secreted proteins, including protein PF963, identified in the culture supernatant of all the nonlytic strains tested. This protein was cut out of a preparative SDS-PAGE gel and subjected to digestion by trypsin. The resulting peptides were analyzed by electrospray ionization tandem mass spectrometry (ESI-MS/MS) on a hybrid triple quadrupole time-of-flight apparatus (QSTAR® XL, Applied Biosystems) according to a standard laboratory procedure (Science and Technology of Milk and Eggs) described in Tarze et al. (2007).

By this analysis, the Inventors identified protein PF963, a secreted bacterial wall peptidase belonging to the NlpC/P60 family. The complete sequence of protein PF963 (SEQ ID NO 36; table I) can be deduced after determination of the complete sequence of the genome of strain CIP103027 by the Inventors.

3-2-2 Fusion of the N-terminal Portion of Protein PF963 with the C-terminal Portion of TRAIL

The presence of a signal peptide at the N-terminal end of PF963 indicates that this enzyme is secreted via the Sec secretion pathway. The sequence of said signal peptide is SEQ ID NO 36. PCR primers were designed to amplify the DNA sequence corresponding to the promoter and to the signal peptide of protein PF963 (FIG. 3). Another pair of primers was designed to amplify the sequence of the human cytokine TRAIL. Only the active extracellular sequence Val¹¹⁴-Gly²⁸¹ was amplified. The primer sequences are indicated in table III below.

TABLE III

| | | | | Nucleotides |
|---|---|---|---|---|
| | | | | hybridizing |
| PCR | | Tm | Total | with the |
| primer | Sequence | (° C.) | nucleotides | matrix |
| P963Fw | ATACATATGCCACCGTGAGCTGCA | 70 | 27 | 18 |
| | CCT | | | |
| | (SEQ ID NO 38) | | | |
| P963Rv | GCAAGCTTTCGGCCTGTGCAAGTG | 71 | 27 | 19 |
| | GTG | | | |
| | (SEQ ID NO 39) | | | |
| TRAILFw | GCAAGCTTAGTGAGAGAAAGAGGT | 70 | 37 | 28 |
| | CCTCAGAGAGTAG | | | |
| | (SEQ ID NO 40) | | | |
| TRAILRev | ACTGCAGTTAGCCAACTAAAAAGG | 70 | 39 | 32 |
| | CCCCGAAAAAACTGG | | | |
| | (SEQ ID NO 41) | | | |

The construction resulting from the fusion between 1) the promoter and the signal peptide of PF963 and 2) the Val¹¹⁴-Gly₂₈₁ sequence of TRAIL was introduced in cloning vector pPK705 (Kiatpapan et al., 2000). The new pFB4:TRAIL expression plasmid is presented in FIG. 4.

3-2-3 Verification of the Genetic Construction

The sequence of the pFB4:TRAIL plasmid was verified (SEQ ID NO 42). The portion corresponding to the fusion protein ranges from nucleotides 8451 to 9070. This portion is translated in FIG. 5 (SEQ ID NO 43): the sequence corresponding to the PF963 peptide signal protein is underlined and the Val¹¹⁴-Gly²⁸¹ sequence of TRAIL appears in bold.

The fusion protein has a sequence of 205 amino acid residues corresponding to a mass of 23,190 Da and an isoelectric point of 9.08. The elimination of the signal peptide leads to a sequence of 171 amino acid residues corresponding to a mass of 19,822 Da and an isoelectric point of 8.60.

Expression and secretion of the fusion protein were verified by Western blot using a commercial anti-TRAIL polyclonal antibody (Pab to TRAIL, Alexis Biochemicals). This antibody recognizes the monomeric form (31 kDa) as well as the dimeric form (63 kDa) of TRAIL in the SuperKillerTRAIL™ preparation (FIG. 6; lane 4). In the supernatant of the two clones of the transformed P. freudenreichii strain carrying the plasmid, a protein of 22 kDa, corresponding to the expected size, was detected by this antibody. This protein was absent in the supernatant of the wild strain.

The expression vector described above has been further modified by the Inventors in order to improve and facilitate both nucleotide sequence cloning and amino acid sequence secretion. To do so, two promoters were selected for their promising properties: as indicated above, the two corresponding proteins, Tuf and BCCP, are major proteins of P. freudenreichii and transcriptomics could confirm an high transcription level of the encoding genes, even under conditions of stress (Leverrier at al., 2004; Falentin et al., 2010). At both extremities of the promoter, multiple cloning sites were added to facilitate subsequent cloning steps.

### IV—Other Embodiments of the Invention

In the examples above, the Inventors made use of a P. freudenreichii signal peptide.

Advantageously, the present invention is implemented using one or more of the P. freudenreichii signal peptides listed in table I above.

Of course, the present invention can be generalized to the use of any propionibacteria signal peptide. The means and methods described in detail above to obtain a recombinant vector enabling the expression and secretion in the extracellular medium of one or more amino acid sequences of interest (the Val¹¹⁴-Gly²⁸¹ sequence of TRAIL in particular) are indeed suitable to the use of any propionibacteria signal peptide.

As an example, a recombinant vector in accordance with the present invention can be constructed using a signal peptide selected from the signal peptides of Propionibacterium acnes, whose genome is available in databases. Table II above gives examples of putative P. acnes signal peptide sequences.

One approach to identifying other signal peptides applicable to the present invention in particular involves aligning sequences in search of Propionibacterium sp. sequences homologous (for example, with roughly 80% homology, preferably at least 85%, 90%, 95% or roughly 98% homology) to signal peptides sequences of a propionibacterium used as a reference (such as P. freudenreichii or P. acnes), or searching for proteins secreted by a given propionibacterium and identifying possible corresponding signal peptides. Current computer software tools make it possible to easily identify putative signal peptide sequences within protein or genomic sequences (for example, the SignalP 3.0 software; Center for Biological Sequence Analysis, CBS; http://www.cbs.dtu.dk/services/SiqnalP/; Emanuelsson et al., 2007).

### REFERENCES

Jan et al. 2002. Cell Death Differ. 9:179-188.
Falentin et al. 2010a. Plos One 5:e11748.
Falentin et al. 2010b. Int. J. Food Microbiol. 144:10-19.
Hervé et al. 2007. Int J Food Microbiol. 113:303-314.
Lan et al. 2007. Apoptosis 12:573-591.
Lan et al. 2007. Br J Nutr. 97:714-724.
Lan et al. 2008. Br J Nutr. 100:1251-1259.
Lacour et al. 2001. Cancer Res. 61:1645-1651.
Lacour et al. 2003. Oncogene 22:1807-1816.
Leverrier et al. 2004. Arch. Microbiol. 181:215-230.
Meurette et al. 2005. Clin. Cancer Res. 11:3075-3083.
Meurette et al. 2006. Ann N Y Acad Sci. 1090:209-216.
Meurette et al. 2007. Cancer Res. 67:218-226.
Kiatpapan et al. 2000. Appl. Environ. Microbiol. 66:4688-4695.
Tarze et al. 2007. Oncogene 26:2606-2620.
Ganai et al. 2009. British Journal of Cancer. 101:1683-1691.
Hu et al. 2009. Cancer Gene Therapy. 16:655-663.
Patch J. A. and Barron A. E., 2002. Curr. Opin. Chem. Biol., 6(6):872-877. Review.
Ashkenazi A et al. 1999. J. Clin. Invest. 104:155-162.
Sträter J. et al. 2002. Gastroenterology 122:659-666.
Sambrook and Russell (2001) Molecular Cloning: A Laboratory Manual (3^{rd} Ed.) Cold Spring Harbor Laboratory Press, Cold Spring Harbor, N.Y.
Emanuelsson et al. 2007. Nature Protocols 2:953-971.
Bougle et al. 1999. Scand. J. Gastroenterol. 34:144-148.
Zarate et al. 2000. J. Food Prot. 63:1214-1221.
Zhang et al. 2010. Cancer Gene Therapy 17:334-343

1. A recombinant vector for expressing and secreting, by a propionibacterium, one or more eukaryotic amino acid sequences of interest, comprising at least:
under the control of at least one suitable promoter,
at least one nucleotide sequence coding for a propionibacteria signal peptide and, in translational fusion with said nucleotide sequence,
one or more nucleotide sequences coding for said eukaryotic amino acid sequence or sequences of interest.

2. The vector according to claim 1, characterized in that said eukaryotic amino acid sequence or sequences of interest have a biological activity of medical interest selected from proapoptotic, anti-inflammatory, immunomodulatory and chemical mediation activity.

3. The vector according to claim 2, characterized in that said eukaryotic amino acid sequence of interest is that of a proapoptotic and/or anti-inflammatory protein or of a biologically active fragment thereof, preferably from human origin.

4. The vector according to claim 3, characterized in that said eukaryotic amino acid sequence of interest is that of a cytokine.

5. The vector according to claim 4, characterized in that said eukaryotic amino acid sequence of interest is that of the proapoptotic protein TRAIL or of the TRAIL C-terminal extracellular domain, preferably the sequence from amino acids 114 to 281 of TRAIL.

6. The vector according to any of claims 1 to 5, characterized in that said signal peptide of propionic origin is a signal peptide of a propionibacterium selected from Propionibacterium freudenreichii, more particularly P. freudenreichii subsp. freudenreichii and P. freudenreichii subsp. shermanii, and Propionibacterium acnes, wherein said propionibacterium is preferentially P. freudenreichii subsp. shermanii.

7. The vector according to claim 6, characterized in that said signal peptide is selected from:
sequences SEQ ID NO 19 to 36 and 45 to 57; and
sequences at least 80% similar to same.

8. A recombinant propionibacterium comprising at least one vector according to any of claims 1 to 7.

9. The bacterium according to claim 8, characterized in that it is a dairy propionibacterium selected from the species Propionibacterium freudenreichii, more particularly P. freudenreichii subsp. freudenreichii or P. freudenreichii subsp. shermanii, P. jensenii, P. thoenii and P. acidipropionici.

10. The bacterium according to claim 9, deposited on Jul. 23, 2009 under number I-4213 with the Collection Nationale de Cultures de Microorganismes (CNCM, Institut Pasteur, 25 rue du Docteur Roux, 75724 Paris Cedex 15, France).

11. A drug comprising an effective quantity of at least one vector according to any of claims 1 to 7 and/or of at least one bacterium according to any of claims 8 to 10, and at least one pharmaceutically acceptable carrier.

12. A method for producing and secreting in the extracellular medium, by a propionibacterium according to any of claims 8 to 10, at least one eukaryotic amino acid sequence of interest, wherein said method comprises at least:
culturing said bacterium under suitable conditions;
recovering the culture medium containing said eukaryotic amino acid sequence of interest; and
optionally, purifying said eukaryotic amino acid sequence of interest.


SEQUENCE LISTING (57 sequences)

SEQ ID NO: 1 - DNA, 99 bp, Propionibacterium freudenreichii
atgtcaaagacactctctcggatcgcatccgtcgcttcggttgccgcgctcgccggcagcatcaccgtcatcgccgggcagaacgcgtccgccgacagc

SEQ ID NO: 2 - DNA, 90 bp, Propionibacterium freudenreichii
gtgaagaacggtctcaagaccctgctcattggtggagtcggcatcgcgacccttgcggtcggcggcgtcggaactgccatcgcagacaat

SEQ ID NO: 3 - DNA, 105 bp, Propionibacterium freudenreichii
atgcgatccaccacgacgaaggcgtttgccggtgtcgctgtgctggcgctggcgcttgctggctgcggctcgaattcgggctcgtccaccaagtcggccgacagc

SEQ ID NO: 4 - DNA, 93 bp, Propionibacterium freudenreichii
atgctcactcgcaagagagtggttgcagcgggagctgccgccaccctgtccctcacggcgtttgccgggttgcagcccgccagcgccgccacc

SEQ ID NO: 5 - DNA, 108 bp, Propionibacterium freudenreichii
atgggattcagggttggccgtcgtcccctcatcggggcagttctcgccgggtcgatggcaacactcgtgggctgttccacctcgggtagcggcagtggagcctccagc

SEQ ID NO: 6 - DNA, 111 bp, Propionibacterium freudenreichii
atgcaggccctccaaggaaggcgccggtcacgacgggtgatggcggccgcggtagcagccctcaccgccatgaccgtgctgccctcccagctcaacgccgttgctgcaccc

SEQ ID NO: 7 - DNA, 90 bp, Propionibacterium freudenreichii
atgtccactggccgcatgaagttcatcaagctggcagttcctgtcatcgttgcctgctgcttgacgccaatggctgccttagctgatgtg

SEQ ID NO: 8 - DNA, 114 bp, Propionibacterium freudenreichii
atggcgatgagggcacgtcacggcgtcgtccggcttggtctggtctgtctcaccgcattggcggtcttcggcacggcaaatgtgtcgggtcaggttgcggtgatggctgagggc

SEQ ID NO: 9 - DNA, 108 bp, Propionibacterium freudenreichii
ttgaaccaggccctgtcgacgatgcgcctgaagatcggcgactccaccaagcgcatccggatcttcttcgtcgtgatggccgtggcgatcaccctgctcgcgggacgg

SEQ ID NO: 10 - DNA, 75 bp, Propionibacterium freudenreichii
atgcgacgtcgcaccacgattgcagccctcgctgctgtcttgagtttcagtcccctggccgcccaggccgcaccc

SEQ ID NO: 11 - DNA, 132 bp, Propionibacterium freudenreichii
atgcctagtcatgcggtgcgggagacgcgggcgaacaagttgcgccggttcctgcggcccaccgttgcccagggcgtgctcggtatcgcgttctgcctcgtggccgccgtcggcgtggtgcagatccgctcc

SEQ ID NO: 12 - DNA, 111 bp, Propionibacterium freudenreichii
atgtcacggattcaactcccccggctgagccggattgcgatcgcagcagcagcttccgctgccctgatcggcaccagcttcatcgccccggccacggcctttgccgcgccg

SEQ ID NO: 13 - DNA, 75 bp, Propionibacterium freudenreichii
ctagccgaccttctcggccttgctggccaggtcgtcggggatcgtgacccccatcagggaggcggccttctcatt

SEQ ID NO: 14 - DNA, 120 bp, Propionibacterium freudenreichii
ttagttgttggggacgaggagggagtggagttcgatgacgtcgagggtgggtgcggtggcggggcgggtgatggtttcggtgccggtgtgtccttgggcggtccaggtgatggtccaggt

SEQ ID NO: 15 - DNA, 60 bp, Propionibacterium freudenreichii
tcagccagttggtgccggccttggcgtcggcggcgtgggatacacgcggaactgggcgcc

SEQ ID NO: 16 - DNA, 114 bp, Propionibacterium freudenreichii
atgttcatttcgcgcttccgtcgtgcggctgcggtcggcctggccgccgtcaccgcattgtccgccactgcctgtagcggttcctcgtcgtcgtccagctcatccgcgagctcg

SEQ ID NO: 17 - DNA, 108 bp, Propionibacterium freudenreichii
atgaagtccgcgacgcgacgcccgctgacgcgctggattgtcgccttcggggtggtgttggtgctggtcatcgccgggtcggtggggctgcatgcctccggtgccctg

SEQ ID NO: 18 - DNA, 108 bp, Propionibacterium freudenreichii
gtgaatcccttcgtcaagacggcgcgcgtggctatcacctcgacgctggtggcaggctcgctcgccactgccagcctcgtgtttgcaccacttgcacaggccgattac

SEQ ID NO: 19 - protein, 33 aa, Propionibacterium freudenreichii
MSKTLSRIASVASVAALAGSITVIAGQNASADS

SEQ ID NO: 20 - protein, 30 aa, Propionibacterium freudenreichii
MKNGLKTLLIGGVGIATLAVGGVGTAIADN

SEQ ID NO: 21 - protein, 35 aa, Propionibacterium freudenreichii
MRSTTTKAFAGVAVLALALAGCGSNSGSSTKSADS

SEQ ID NO: 22 - protein, 31 aa, Propionibacterium freudenreichii
MLTRKRVVAAGAAATLSLTAFAGLQPASAAT

SEQ ID NO: 23 - protein, 36 aa, Propionibacterium freudenreichii
MGFRVGRRPLIGAVLAGSMATLVGCSTSGSGSGASS

SEQ ID NO: 24 - protein, 37 aa, Propionibacterium freudenreichii
MQALQGRRRSRRVMAAAVAALTAMTVLPSQLNAVAAP

SEQ ID NO: 25 - protein, 30 aa, Propionibacterium freudenreichii
MSTGRMKFIKLAVPVIVACCLTPMAALADV

SEQ ID NO: 26 - protein, 38 aa, Propionibacterium freudenreichii
MAMRARHGVVRLGLVCLTALAVFGTANVSGQVAVMAEG

SEQ ID NO: 27 - protein, 36 aa, Propionibacterium freudenreichii
MNQALSTMRLKIGDSTKRIRIFFVVMAVAITLLAGR

SEQ ID NO: 28 - protein, 25 aa, Propionibacterium freudenreichii
MRRRTTIAALAAVLSFSPLAAQAAP

SEQ ID NO: 29 - protein, 44 aa, Propionibacterium freudenreichii
MPSHAVRETRANKLRRFLRPTVAQGVLGIAFCLVAAVGVVQIRS

SEQ ID NO: 30 - protein, 37 aa, Propionibacterium freudenreichii
MSRIQLPRLSRIAIAAAASAALIGTSFIAPATAFAAP

SEQ ID NO: 31 - protein, 25 aa, Propionibacterium freudenreichii
MKRRTLLGTLGIMGLSVPLAACSSK

SEQ ID NO: 32 - protein, 40 aa, Propionibacterium freudenreichii
MVTGGNDMPSKRITTWPGISALSALIAGMLLAPLPVAADG

SEQ ID NO: 33 - protein, 20 aa, Propionibacterium freudenreichii
MAMVMASLAMFGASRASAAD

SEQ ID NO: 34 - protein, 38 aa, Propionibacterium freudenreichii
MFISRFRRAAAVGLAAVTALSATACSGSSSSSSSSASS

SEQ ID NO: 35 - protein, 36 aa, Propionibacterium freudenreichii
MKSATRRPLTRWIVAFGVVLVLVIAGSVGLHASGAL

SEQ ID NO: 36 - protein, 36 aa, Propionibacterium freudenreichii
MNPFVKTARVAITSTLVAGSLATASLVFAPLAQADY

SEQ ID NO: 37 - protein, 572 aa, Propionibacterium freudenreichii
MNPFVKTARVAITSTLVAGSLATASLVFAPLAQADYSPLAATATVNVRQGPDTSSSVLATLSSGDTVTQRGAEQDGWLPITYNGANAWIQAQYVASTTAATQKDQISTAELTADAYVRTAANANAWVLGTAHTGDKVGITGQASGDYTPVNFYGRAGWIATKLLSAADASVTSIKITTAISSDYLWVRGGESTAAQSIGMLYPGDRVDVTGDPVGGWVPINFNGKTAFVAANYSRYLTDPTVVTLSTKTDVTNKDTATSTGTDSSTAGGSTATTPTTTAPTTTAPATKPTTTPPATTQAAASTKYTTADVNVRVGPGIDQQPVTVLKENSQVAATGKTSGDWTEVSYDGASRWISSQYLSDTKQAEAPAPAPAPDPTPAGPTGSRWTTAALNAYGSSTQPKPATTVVPEGTQVELTGKQADGRSEYTWNGTTYWSATEYLGTNAPATNTSANTAKPGANAVETAINFAMSKLGGPYVWGGTGPVGYDCSGLMQAAYAAAGVTLPRVTWDQVNAGKQVSVGDLQPGDLVFFYDNGHVGMYIGNGNIVNALNEDAGIVVTPISYMPISAAVRIA

SEQ ID NO: 38 - DNA, 27 bp, artificial
  Primer P963Fw
atacatatgccaccgtgagctgcacct

SEQ ID NO: 39 - DNA, 27 bp, artificial
  Primer P963Rv
gcaagctttcggcctgtgcaagtggtg

SEQ ID NO: 40 - DNA, 37 bp, artificial
  Primer TRAILFw
gcaagcttagtgagagaaagaggtcctcagagagtag

SEQ ID NO: 41 - DNA, 39 bp, artificial
  Primer TRAILRv
actgcagttagccaactaaaaaggccccgaaaaaactgg

SEQ ID NO: 42 - DNA, 9073 bp, artificial
  Sequence of the plasmid pFB4TRAIL
ggtcgactctagagggatccggcggaacttcacgtcctggcggtggagttggcgggcgcgttccagccgttcctccagcacggtgatccgggcctccagacgctcacgctcaccctgctccaggtgccgggtcaccgtcaccgtccgcaccggccgggcctcggcctgggcggcccggcgttcctcactggcccgcttccggcaatcgtcggaacaccacacccggggccgaccccgcccaccgtgggcctccaccggcgccccgcagtggggacacgcccgcagcgccgacgcatcctcatccaaggccatcaccgggtcggaatccatacccgaaaccatatcgtccggacgatgaactgcgccagacagctaagaatgcacgaggtgtgtctccgattctcaggaaacgctcagcattttccgagacgttcggcgcacgcacacacccccacaagaaccgacccgcccagcatccgccgacacgtcgatccgcacccgcgatgggctggccgaggccgactacgaccgctagtcagcacctgcgctgatctaccgtcgccctgaccgactctcccgtcgggattgtcgccggccgctgccagcatggacctgcggccccgccccctcgccctgcaactcgagggaggcggggccgtccaccccccacaccaccccgacaccgtgatgcgcccatgtcgcctaacgggttgcccgacctccccgacatcaagaaaacctgacaccgtcgccgcaagcgctacactgactactagtagtcaggaggtgcgtgatgaccatcgccacatcggtgaaactctccgaagagaccggccgcaaactcgatgaactagcccgggccaccgggcgatccaagtcctactacctgcgcgaggccatcgaggaccacatcgaccagatggtccacgactacgccatcgcccgactcgccgacgacgtgcgagccggccgggccgccacctacagcgccgacgaagtggaccagatccttggcctggacgattgagtacaccgaccccgccgtcaaagcactgcgcaaactcgaccgagcccaggcccgccgcatcaccgcctacatacgtgagctcaccggcctggacgatccccaccaacgcgggaaaggaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgtaatgtgagttagctcactcattaggcaccccaggctttacactttatgcttccggctcgtatgttgtgtggaattgtgagcggataacaatttcacacaggaaacagctatgaccatgattacgccaagctgtggcgtacaccgtcgcctcggtcggcccgtagagattggcgatcccgaccgcagcaccaccgagaacgtccccgacgtggccgaccagcccgtcatcgtcaacgcctgaccgcggtgcggacaggccgtgtcgcgaccggccgtgcggaattaagccggcccgtaccctgtgaatagaggtccgctgtgacacaagaatccctgttacttctcgaccgtattgattcggatgattcctacgcgagcctgcggaacgaccaggaattctgggagccgctggcccgccgagccctggaggagctcgggctgccggtgccgccggtgctgcgggtgcccggcgagagcaccaaccccgtactggtcggcgagcccgacccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgacgccccgggcctaccgctgacacgcaaccccgcaccctcggccaagacgtcacacaccacccgccccaccgagcactgaggatgtcaactcgcccgagccggcctgccggccgtcttacgggttgtcttggcgggcggggtgtctttgccctggcccagcagccccacgatctcccgcagcgtgtcggcggtggcggcgtcccgggccgcctgacgctccgcctccgccctggcctgctcggctgcctgcgcccgatcctccgcggcggcggcctgctccctcgcctcggccagctcgccggtcagggcctcgacccgggcctgcacctgccccaggcgcgcctccgcctcctgctgcacctgctcggcccgggcctccgcctggtcccgggccgcctcggcctcggcccggtgctgatccgccagggccgcctcggccaccgcttcggcctgcccatccaccgcctgctcggcccgagccccgaactcctcgcgggccgcatcactcgcctgacgccacgccgccgcccacaccagacccaacggctccgacagatccggcggggccggcgtctggaccgacgccgagacgtcgcgcaggaaccccgccgcagcgtcggtggagcaccccgcctccgccttcaacgaccgcaccgtcacccgccgacccgcaccgctcaaccgcgcataggccgccgccaaccttgacccattcgactccatgacccaccctcccattctgtaccctgtacctgttcctaggtacgttcctaatgtacctcaccggatgcagaacccgcaacccccctcacactccccctgcacggggcccgccccctgcacccccgctgccgcgcccgctcctgcgtcgcggccttgcccctgcccaacgccgggccggcgggcagcccaccagaggctctgtgagacgtcggcgcccccgtccacctaccctaaagaccaaccggccgtggaaacgtctgtgaggagccttgtaggagttcccaggacaagccagcaaggccgggcctgacggcccggaaaggaagtcgctgcgctcctacgaagaagcccctctggggacccccagaccccggaactatctgatttggtttagcggcgtacttccgtcataccggaatttatggcatgctgtggtcatggcgacgacgacggtcgatgagcagtgggagcaggtgtggctgccccgctggcccctggcctccgacgacctggcagcgggcatctaccggatggcccgcccctcggcgctgggggtccgatacatcgaggtcaacccccaagccatcagcaacctcctcgtggtcgactgcgaccaccccgacgctgccatgcgcgccgtctgggaccgccacgactggctgcccaacgccatcgtcgagaaccccgacaacggccacgcccacgccgtgtgggccctggaagcagccatcccgcgcaccgagtacgcccaccgcaagcccatcgcctacgccgccgccgtcaccgagggcctgcgccgatccgtcgacggagacgcctcctacgccggcctgatcaccaagaaccccgaacaccccgcctggaacaccacctggtgcaccgaccacctctaccggctggccgagctcgacacccacctggatgccgccggcctcatgcccgccccctcctggcgacgcacccgccggcgcaaccccgtcggcctgggccgcaactgcgccatcttcgagaccgcccgcacctgggcctaccgcgacgcccgccgcatccgacaacgccacgaatacccgaccgccgaggactcggccgacctgcacgccgtcatcgcctccaccgtcgaggcgctcaacgccggctacagcgaacccctgccggcccgcgaggccgccggcatcgccgccagcatccaccgatggatcacccaccgtttctacggctggatcgactcccacaccgtcaacgaggccactttctccaccatccagagctacagaggacacaagggagccggcaaggctcgtcctcgtgcccgccgtgctgcttctatcaccgattgggaggcatgatggctgacgtccagcaccgcgtgaagcgtcggggcacggcccgcgaggccgcagaacgtgtaggggcctccatccgaaccgcccagcggtggacctccatcccccgtgaggaatggatcactcagaaggccgtcgagcgtgaggagatccgggcctacaagtacgacgaggggcacacgtggggcgagacctcgcgccacttcgggatcgcgaagaccaccgcccaggagcgggcccggcgggctcgaagggagcgggcggccgaagcggagaaggctgccgaggaggccgaggccgcgctgcgtccgacactcttcgagggccaggagcaaggttctgcatgagcaaccccgagtcctcgggtagaccgtctggcccgacgttaagcatggctgaagcggcccgtgcctgtggggtttcagtgtccacggtgaggcgtcaccgtgatgccctggtggcccacggtgctacccgtcatgacgcgtcatgggtgatacccctatcagcgttgatttcatgcggtttgatgccccgggtgacaccccctgatgccccgtcacccaataacgtggcgcctgccatgacgtcccacggtgacgcccccctgacgggggaagtccaagagctgcgcgagcgactggccaacgctgagcatcgagccgagctagcagtagaggttggggacgacgtctcggcgactccggagaacaccaagtcagggtctcatgagtgtgcgatagcttgagctgtctaccaatctggatatagctatatcggtcgtttgtgtctgattcgccagtgagccaacggcgggggcgacacgcggtggcgaaaccccctggcagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgccaccgtgagctgcacctgaatcagctgagaatgccctgaaccctcaaacccgcaccctgaacctcaaccttgcgttgaacccgaggggtgcgggttggacgcgcaccccgaccactgcaccccgcggggcgcccctgtgacgaccatggtctgattcacgcctgaaatcactccccgccggggtggagaaccacgtcaacgcggccgtggatcacatcgggcgtcgaaaaacaaccccccatttccccaaccctcaacctgatcctgcactgttgtcgggtttgctgagagccgcctaagctgccgcacgttgtcccagttggggcgtggcctgctgcatacggggccgggaaagacgcctcacctgggatgacgcggaccattggacacggcctttccggccgcgggaaggaccagacgtgaatcccttcgtcaagacggcgcgcgtggctatcacctcgacgctggtggcaggctcgctcgccactgccagcctcgtgtttgcaccacttgcacaggccgaaagcttagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgca

SEQ ID NO: 43 - protein, 205 aa, artificial
  Fusion protein sequence encoded by pFB4TRAIL
MNPFVKTARVAITSTLVAGSLATASLVFAPLAQAESLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 44 - DNA, 1719 bp, Propionibacterium freudenreichii
gtgaatcccttcgtcaagacggcgcgcgtggctatcacctcgacgctggtggcaggctcgctcgccactgccagcctcgtgtttgcaccacttgcacaggccgattactccccccttgcggccaccgccacggtgaatgtgcgccagggccccgacacctccagctcggtgctggcgacgctctcgtcgggtgacacggtcacccagcggggagccgaacaggacggctggctgccgatcacctacaacggcgccaacgcgtggatccaggcgcagtacgtggcctccaccacggccgccacccagaaggaccagatctccaccgccgagctcacggccgatgcctatgtgcgcaccgcggccaatgcgaacgcctgggtgttgggcacggcccacaccggcgacaaggtgggcatcaccggccaggccagcggcgactacacgccggtgaacttctacggccgcgccggttggatcgccaccaagctgctcagcgcggccgacgcctcggtgacatcgatcaagatcaccaccgccatctcgagcgactacctgtgggtgcgtggtggcgagagcaccgcggcccagtccatcggcatgctctacccgggtgaccgggtggacgtgaccggcgatcccgtcggcgggtgggtgccgatcaacttcaacggcaagaccgccttcgtggccgcgaactactcgcgctatctcactgatcccaccgtggtcacgctgtcgacgaagaccgacgtcaccaacaaggacacggccacctcgacgggcaccgattcctcgacggccggcggctcgaccgccaccaccccgaccaccacggcgccaaccaccaccgctcctgcaaccaagccgacgaccacgcccccggccacgacgcaggccgctgcgtccacgaagtacacgacggccgacgtcaacgtgcgcgtgggacccggcatcgaccagcagccggtgacggtgctcaaggagaactcgcaggtggccgccaccggcaagacaagtggcgactggaccgaggtcagctacgacggcgcctcgcgctggatcagcagccagtacctctcggacaccaagcaggccgaggcacccgccccggcacctgcgcccgatccgacgcccgccggccccaccggaagccggtggaccacggcggcactgaacgcctatggcagctccacccagcccaagccggccaccacggtggtgcccgagggcacccaggtggaactgaccggcaagcaggccgatggacgctcggagtacacgtggaacggcacgacctattggtcggccaccgaatacctcggcaccaatgcgccggccacgaacacctcagcgaacaccgccaagccgggcgccaacgcggtggagacggcgatcaacttcgcgatgtcgaagctcggtggcccctatgtctggggcggcaccggtccggtgggctatgactgctccggactgatgcaggccgcgtacgcggcggccggcgtcaccctgccgcgcgtcacctgggaccaggtgaatgccggcaagcaggtgtcggtcggcgacctgcagccgggcgacctggtgttcttctatgacaacggccacgtgggcatgtacatcggcaacggcaacatcgtcaacgccctcaacgaggacgccggcatcgtggtgaccccgatcagctatatgccgatctcggctgccgtccggatcgcctga

SEQ ID NO: 45 - protein, 26 aa, Propionibacterium acnes
MSKVVASAIAGALSLTSAGGLTMVQA

SEQ ID NO: 46 - protein, 30 aa, Propionibacterium acnes
MRKAIVTPVAVLAVLVMALTGCGQKNQSGG

SEQ ID NO: 47 - protein, 29 aa, Propionibacterium acnes
MASTPRRRWAWVLLLVVASLVIVGVYRKA

SEQ ID NO: 48 - protein, 26 aa, Propionibacterium acnes
MSSMKGLSLVLATSFMLSFSPGSSFA

SEQ ID NO: 49 - protein, 44 aa, Propionibacterium acnes
MEHRYGASQVSGSAPRRGRGVAFAAITGAILLGTVASVDPGAQA

SEQ ID NO: 50 - protein, 27 aa, Propionibacterium acnes
MSSMKGLSLVLATSFMLSFSPGSSFAS

SEQ ID NO: 51 - protein, 47 aa, Propionibacterium acnes
MPHSDQPTSKRVMSAPRRRMPGWVPVTVGIAVVVIVVVAVIVSSLRS

SEQ ID NO: 52 - protein, 32 aa, Propionibacterium acnes
MFGTPSRRTFLTASALSAMALAASPTVTDAIA

SEQ ID NO: 53 - protein, 26 aa, Propionibacterium acnes
MKINARFAVMAASVAVLMAAAPIAQA

SEQ ID NO: 54 - protein, 50 aa, Propionibacterium acnes
MYPVHLPLRNESEFSFRAHNHGGTVPSRLTRRSVLATGAVALPMTAAACA

SEQ ID NO: 55 - protein, 27 aa, Propionibacterium acnes
MRHMRPLIALSLAGLMTLSACGEDVAA

SEQ ID NO: 56 - protein, 31 aa, Propionibacterium acnes
MNRTLKVAAVGAIAILCLAACSDPGSDSAQS

SEQ ID NO: 57 - protein, 24 aa, Propionibacterium acnes
MEKSSFAAANMTIMSEPTTPTSQA